(19) United States
(12) Patent Application Publication
(10) Pub. No.: US 2008/0215014 A1
(43) Pub. Date: Sep. 4, 2008

(54) Manually activated flow/no flow medical slit valves and related methods

(76) Inventor: Greg Nordgren, Lehi, UT (US)

Correspondence Address: Lynn G. Foster, L.C., Suite C3, 65 N Street, Salt Lake City, UT, 84103, US

(21) Appl. No.: 11/713,320

(22) Filed: Mar. 1, 2007

(51) Int. Cl. A61M 5/00

(52) U.S. Cl. 604/247

(57) ABSTRACT

Slit valve technology is disclosed comprising medical slit valves and related methodology, the slit valves being either normally open or normally closed. A slit or slits are disposed in membranes or diaphragms spanning across the lumen of a cannula of elastomeric material with memory. Each slit valve is closed and opened by manual manipulation of an external actuator radially disposed outside the cannula. The elastomeric cannula are shown to be in the shape of a sleeve, which is connected to a housing of a medical valve assembly, although the present invention is not so limited.

### FIELD OF INVENTION

The present invention relates generally to control of liquid flow in a medical cannula and, more particularly, to novel manually activated flow/no flow medical slit valves and related methods. The present invention is directed to selective valving of fluid flow through a displaceable slit valving member with memory, the slit valving member being physically displaced by a manual control element or actuator to accommodate unidirectional or bi-directional flow, along a hollow medical cannula, such as a catheter tube or needle. The slit valving member may be normally open or normally closed.

### BACKGROUND

In the past, slit valves have traditionally been used in the side walls of otherwise closed indwelling catheter tubes to infuse or aspirate fluid. Use of such side wall slit valves has been directed to infusion and aspiration of liquids in the cardiovascular systems of medical patients, infusion and aspiration of fluids in the respiratory systems of medical patients, and infusion and aspiration in other body cavities.

Disadvantageously, sometimes the central passageway within an indwelling catheter tube, which comprise one or more side wall slit valves, is partially or totally occluded when the slit valve is flexed inwardly to an open position. Also, interference can occur between the outwardly flexed lips of the slit and the wall of the body cavity in which the catheter tube and indwelling slit valve are disposed, which either prevents the slit valve from opening or undesirably limits the extent to which it is permitted to open or prevents or unduly limits flow.

Outdwelling slit valves have been proposed in the past. For example, see U.S. Pat. Nos. 5,201,722 and 5,984,902, which disclose transversely directed disc-shaped slit valve diaphragms each having a normally closed central slit, the axial flexural displacement of which is mandatorily constrained by abutment structure fore and or aft of each transverse disc-shaped diaphragm.

Medical slit valves of the past have been limited to normally closed configurations, as opposed to normally open versions, because pressure differential cannot be used to close a normally open slit valve.

The prior art does not propose use of a manually displaceable internal or totally or partially concealed actuator by which an elastomeric slit valve element of a valve assembly is displaced manually by a medical provider between open and closed positions.

### BRIEF SUMMARY AND OBJECTS OF THE INVENTION

In brief summary, the present invention overcomes or alleviates problems of the past in controlling fluid flow to and from medical patients utilizing slit valves. The technology of this invention comprises medical slit valves and related methodology, the slit valves being either normally open or normally closed, each of which is disposed in membranes or diaphragms spanning across a lumen of a cannula of elastomeric material with memory. Each slit valve is closed and opened by manual manipulation of an external actuator radially disposed outside the cannula. Typically, the elastomeric cannula may be in the shape of a sleeve, which is connected to a housing of a medical valve assembly, although the present invention is not so limited.

With the foregoing in mind, it is a primary object of this invention to overcome or alleviate problems of the past in respect to medical slit valve technology.

Another paramount object is the provision of novel medical slit valve technology, including apparatus and method, by which a slit valve may comprise either normally closed slit or normally open slit, when at rest, disposed in a membrane or diaphragm spanning across a lumen defined by a cannula of elastomeric material with memory such that manual manipulation of an outside actuator deflects the cannula and thereby displaces the slit between closed and open positions or between open and closed positions.

These and other objects and features of the present invention will be apparent from the detailed description taken with reference to the accompanying drawings.

### BRIEF DESCRIPTION OF THE DRAWINGS

FIG. 1 is a perspective of a valve assembly comprising hub, a housing in which a slit valve, embodying principles in accordance with the present invention, is contained, a distal adapter and an outside slit valve actuator;

FIG. 2 is a fragmentary cross section taken along lines 2-2 of FIG. 1;

FIG. 3 is a cross section taken along lines 3-3 of FIG. 2, showing the normally closed slit valve in its closed position;

FIG. 4 is a fragmentary cross section similar to FIG. 2, but showing the normally closed slit valve in its open position;

FIG. 5 is a cross section taken along lines 5-5 of FIG. 4;

FIG. 6 is a fragmentary cross section similar to FIG. 5 of a second embodiment of the present invention;

FIG. 7 is a cross section taken along lines 7-7 of FIG. 6, showing the normally open slit valve in its open position;

FIG. 8 is a fragmentary cross section similar to FIG. 6, but showing the normally open slit valve in its closed position;

FIG. 9 is a cross section taken along lines 9-9 of FIG. 8;

FIG. 10 is an enlarged fragmentary cross section of another, single housing embodiment;

FIG. 11 is an enlarged fragmentary cross section similar to FIG. 11 showing an elastomeric cannula comprising a normally open slit diaphragm with a hypodermic needle passing through the slit;

FIG. 12 is a diagrammatic representation of a valve assembly of this invention placed between an IV battle and an IV tube in lien of a conventional clamp;

FIG. 13 is a perspective of a catheter mechanism comprising two proximal valve assemblies; and

FIG. 14 is a perspective of a dual slit valve catheter mechanism comprising a proximal and a distal slit valves.

### DETAILED DESCRIPTION OF THE ILLUSTRATED EMBODIMENTS

The present invention relates to novel slit valve technology and its use in one way and two way flows of fluid to and/or a medical patient. The novel technology has application to use in conjunction with the human respiratory system, the human circulatory system, and other body cavities. Typically slit valves of the present invention are placed inside of a proximal valve assembly located out dwelling of the medical patient. The slit valve technology of the present invention relates to both normally closed and normally open slit valves, i.e. the slit is closed and open, respectively when at rest. The normally closed versions of the present invention may, under some conditions, accommodate pressure differential flow through the slit. However, the substance of the present invention deals with physically opening and closing the slit valve using an outside or external actuator and physical force applied by the medical provider, independent of the pressure differential being imposed upon an internal membrane or diaphragm containing the slit or slits. The preferred materials for forming the slit valves of this invention comprise silicone rubber, polyurethane and other deformable natural and synthetic elastomeric materials.

In other words, the medical attendant manipulates the outside actuator so as to mechanically or physically displace or distort the internal diaphragm, changing the slit therein from open to closed or closed to open, as the case may be. This approach allows higher flow rates and greater volumes through the slit, without valve pressure restrictions. It also allows low pressure infusions, such as a gravity drip, to be unrestricted by the valve, when the slit is open, because a pressure differential of a given threshold value must not first be reached.

The normally closed slit valve technology of this invention allows infusion of fluid into the medical patient with either a syringe or a pump because the pressure values accommodated by the present invention in the manner indicated would be higher than current slit valves on the market. The present valves allow pressures to exceed both external and body pressures thereby making the present invention safer and more reliable.

Because the normally closed versions of the present invention are opened by external force for low pressure infusion, accommodating high volume flow, the present invention is available for dialysis and other important medical applications. Slit valves in accordance to the present invention can be flushed while the valve itself retains its fluid column and maintains lumen patentcy.

While the present invention accommodates use at the proximal end of a catheter tube, it may also comprise an intermediate and a distal catheter valve. This ensures total catheter lumen fluid control. The distal valve prevents body fluids from entering the lumen and, if a proximal valve according to the present invention is also used, it stops outside air from entering.

Normally open slit valves embodying the present invention may be utilized on the interior of a proximal valve assembly, but the present invention is not limited to use at that location. It is believed that a normally open slit valve for fluid flow to and from a medical patient has not heretofore been proposed. Use, among others, may be in two different ways. First a normally open slit valve in accordance with the present invention may be used at the proximal end of an over-the-needle IV catheter. When the over-the-needle IV catheter is assembled, the needle is inserted through the normally open slit of the slit valve diaphragm in accordance of the present invention thereby avoiding any tendency for the diaphragm defining the slit to take a set, which set might thereby make closure of the slit thereafter difficult if not impossible after the needle is withdrawn. After needle removal, the normally open slit can be closed in the manner described hereinafter. Once the IV set is connected to the proximal end of the catheter tube and is ready for use, the slit valve may be returned to its normally open position accommodating IV flow to the patient. When it is time to change from one IV set to another, the normally open slit valve is again closed by manual manipulation by the medical attendant of the outside actuator until the change in IV sets has been accomplished.

In addition to the foregoing, normally open slit valves in accordance with the present invention may be utilized in conjunction with any catheter product where external clamps are currently being used. Thus, normally open slit valves in accordance with the present invention may replace the bulky clamps currently being used, whereby potential damage by the bulky clamps to the medical tubing is prevented. In addition, a normally open slit valve in accordance to the present invention can be manually closed when the product is installed on the patient but not being used, thereby creating a safer condition for the patient. When the valve is closed, the associated catheter may be flushed through the slit valve without physically opening the slit valve. To the contrary, conventional clamping devices require that the clamp be opened, the tube flushed and then the clamp closed, with hope that blood did not enter the distal end of the catheter during the process.

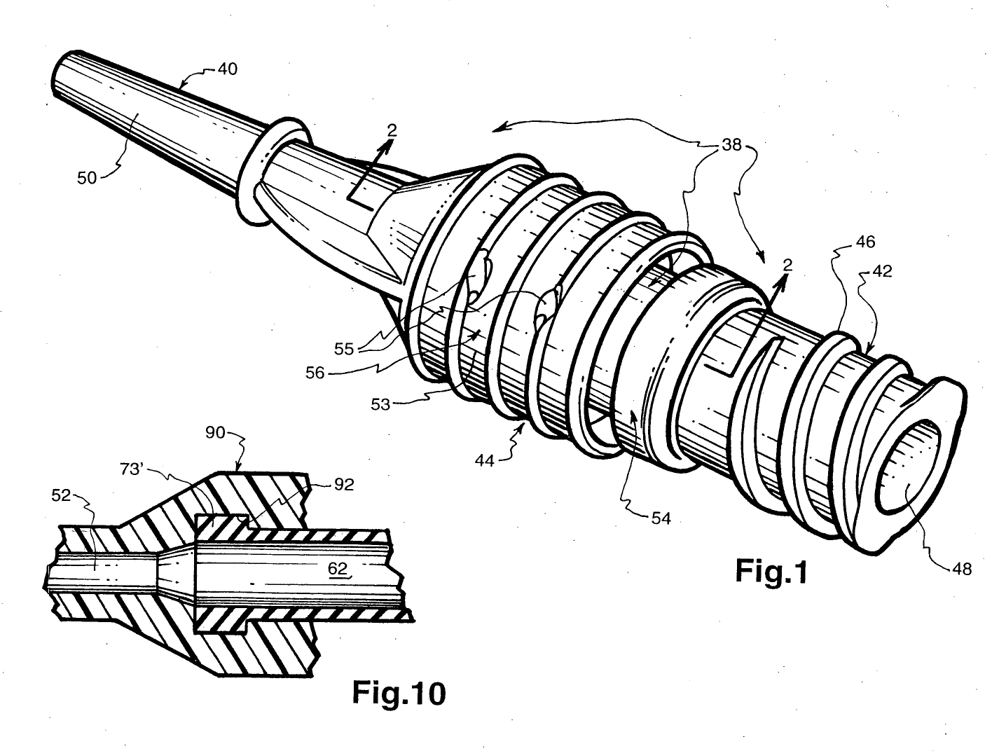

Reference is now made to the drawings, wherein like numerals are used to designate like parts throughout. Slit valves of this invention may be used for infusing, aspirating or both and may be any one of many configurations. Each of the slit valves shown in FIGS. 1-10, which are merely representative, comprises a slit placed in a membrane or diaphragm spanning across a lumen of an elastomeric cannula with memory for use with a medical patient to selectively accommodate fluid flow to and from a medical patient. The elastomeric cannula and diaphragm are preferably formed of one piece construction.

FIG. 1 depicts one slit valve version in accordance with the present invention. The slit valve assembly 30 of FIG. 1 comprises a concealed diaphragm located within a housing, generally designated 38. The housing 38 comprises distal and proximal housing parts 40 and 42. The slit valve diaphragm within housing 38 accommodates selective fluid flow through a slit or slits into and from a body cavity of the patient. The slit in the diaphragm is opened and closed by an outside actuator 44. While, in some instances, the body cavity would be a cardiovascular vein, where the fluid flow comprises liquid displacement, liquid displacement involving other body cavities is contemplated, as is gaseous flow from and to other body cavities.

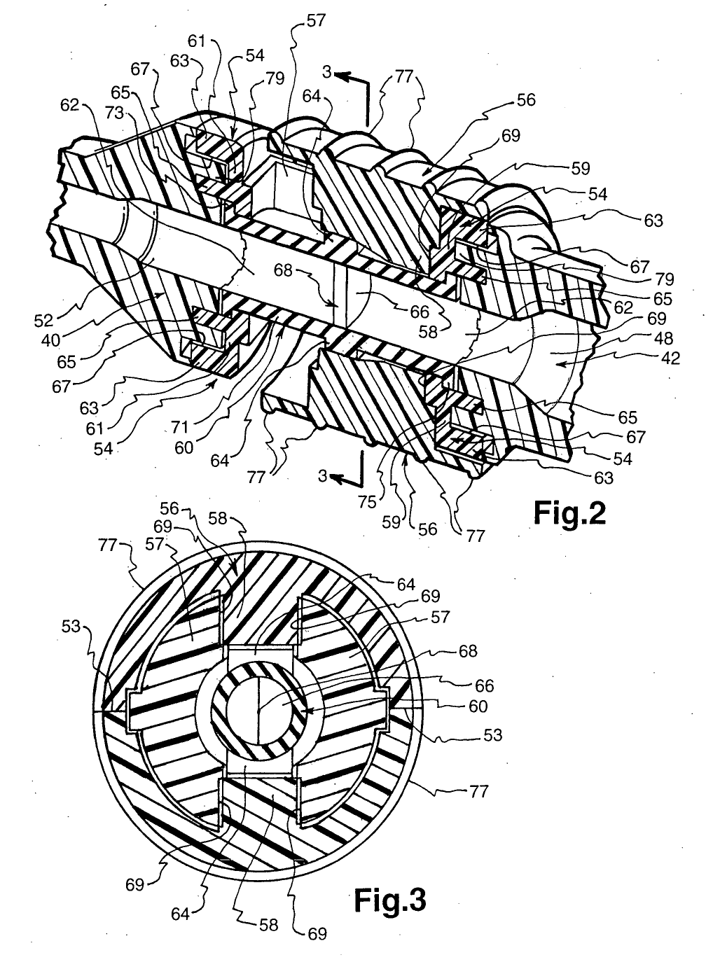

The exterior of the proximal housing part 42 comprises an exposed luer lock thread 46 and a hollow interior 48 to accommodate selective fluid flow. Thread 46 accommodates luer lock threaded connection with a luer lock fitting, for example, located at the distal end of hollow proximal tube, such as an IV tube connecting to a container of IV solution, in a conventional manner. Distal housing part 40 comprises a tapered elongated tip 50 sized, for example, to accommodate a press fit overlapping connected relationship with the proximal end of an IV catheter connected to the patient. The interior of the distal housing part 40 comprises the hollow passageway 52 (FIG. 2) accommodating selective fluid flow.

The proximal and distal ends of housing parts 42 and 40 are respectively connected to a central housing member 54. Carried on the exterior of the simple housing member 54 is a reciprocable manually-operated actuator, generally designated 56.

The central housing number 54 comprises axially extending supports 57 formed as one piece with and bridging between annularly extending, bifurcated proximal and distal ends 59 and 61. The bifurcated ends 59 and 61 comprise spaced annular flanges 63 and 65, which extend in an axial direction. Flanges 63 and 65 define an annular groove 67 there between, for purposes of connection to proximal housing parts 40 and 42. Specially, an annular tongue 79 is inserted between flanges 63 and 65 at each end and bonded or otherwise secured there against removal. The central housing member 54 comprises openings 69 through which actuator 56 extends. More specifically, annular 10 of actuator 56 extends through the openings 69 of the central housing member 54.

An axially directed cannula, generally designated 60, is positioned concentrically within the housing 54 and the actuator 56. Cannula 60 is of elastomeric material with memory, such as silicone rubber, and comprises a central longitudinal axis which is common to the longitudinal axis of the passage ways 48 and 52 of the housing parts 40 and 42.

While the elastomeric cannula 60 does not mandate a cylindrical or annular configuration, the elastomeric cannula 60 shown in the Figures is configurated in the form of a sleeve 71, having, with the exceptions mentioned below, a uniform inside diameter and a uniform outside diameter, such that the sleeve wall 71 is of uniform thickness. Thus, the wall 71 defines a lumen 62 of substantially uniform diameter and, in the configuration illustrated, the diameter of lumen 62 is substantially the same as the adjacent diameters of passageways 48 and 52. The wall 71 terminates distally and proximally in outwardly directed radial flanges 73 and 75. Flange 73 is axially compressively retained between central housing member 54 and distal housing member 40 with sufficient compressive force that inadvertent separation is prevented.

Flange 75 is axially compressively held against inadvertent separation by contiguous compressive engagement between and by central housing member 54 and proximal housing member 42.

The uniform thickness of wall 71 is interrupted at the center of elastomeric cannula 60 by an outwardly extending radial directed cam follower 64, which is formed as one piece with the elastomeric cannula 60 and is selectively displaced by the cam 58 as explained hereinafter.

Also, the lumen 62 is normally closed at central location of the wall 71 by diaphragm or membrane 66, which comprises a normally closed slit 68. The diaphragm or membrane 66 is formed as one piece with the wall 71, as are flanges 73 and 75.

FIG. 2 illustrates the normally closed slit 68 of diaphragm 66 in its at rest, closed condition wherein the wall 71 at its center is unstressed and the cam 58 and cam follower 64 are axially spaced one from the other. This condition is illustrated in FIGS. 2 and 3.

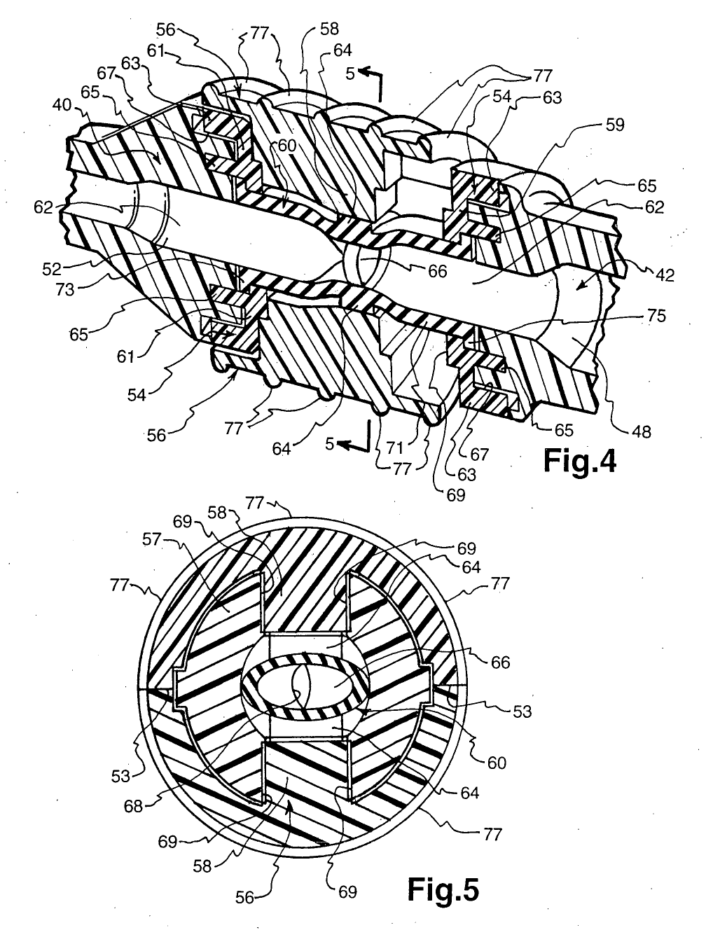

When the medical attendant desires that fluid be caused flow across the diaphragm 66, the medical attendant grasps the ribs 77 of the slidable actuator 56 and displaces the actuator, relative to the elastomeric cannula 60 from right to left as shown in the FIGS. 2 and 4, in other words from its proximal position of FIG. 2 to its distal position of FIG. 3. Cam 58 of the actuator 56, as a consequence of the displacement, to ride over and inwardly compact the cam follower or protrusion 64. This causes the diaphragm 66 to be compacted and distorted as its diameter is reduced by the described force applied by the cam 58. This displacement and distortion of the elastomeric cannula 60 and the diaphragm 66 is shown in FIGS. 4 and 5. The consequence is to flex into an open position the lips of the diaphragm defining the slit 68 as a consequence flow of lumen 62 through the open slit 68 is accommodated. See FIG. 5. With the slit caused physically to be open fluid flow through the slit across the diaphragm is accommodated.

When the medical attendant desires fluid flow across the diaphragm 66 to discontinue, the medical attendant simply moves the actuator 56 left to right from the position of FIG. 4 to the position of FIG. 2. Thus, the configuration of the present invention illustrated in FIGS. 1-5 comprises a normally closed slit valve which may be physically opened by selective manual manipulation by a health care provider of the controller or actuator. Thereafter, the valve is manually closed by reverse manual manipulation of the actuator or control.

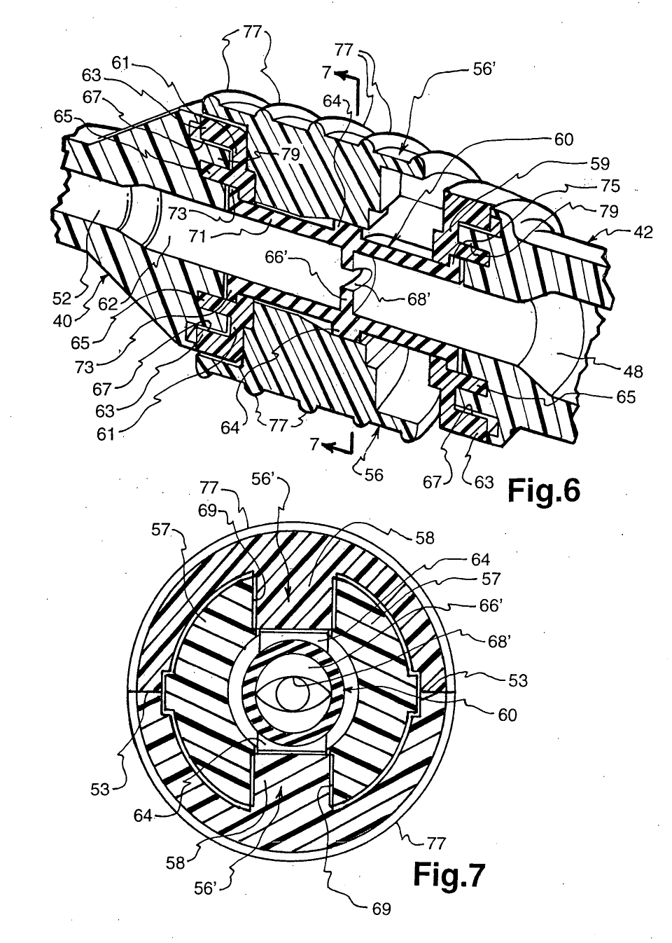

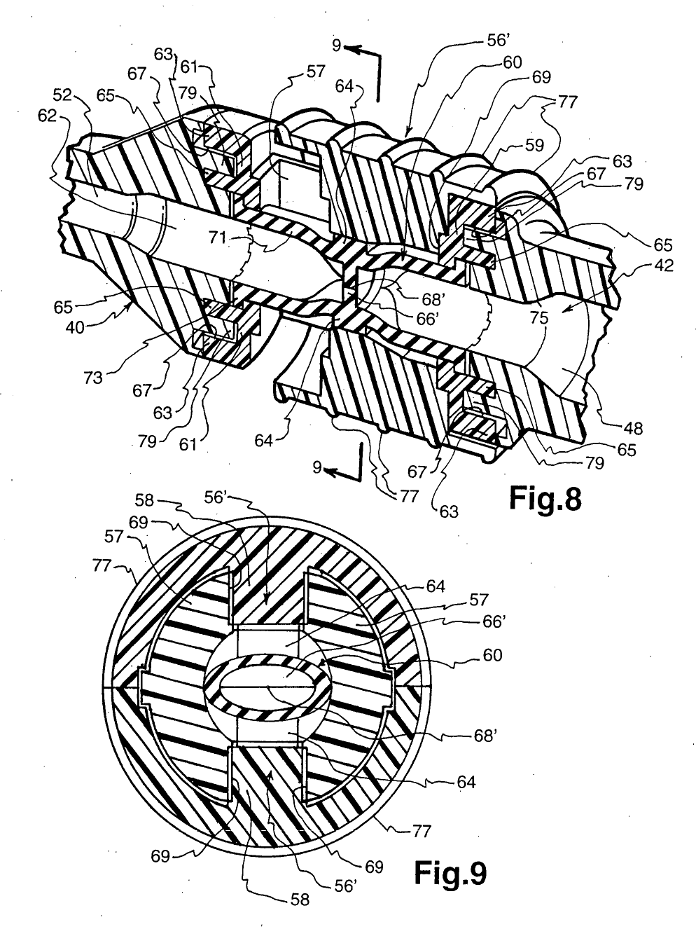

With references now made to the embodiment illustrated in FIG. 6-9, by the health care provider the components of which are identical to the embodiment of FIGS. 1-5, except as mentioned below. The slit 68′ of the diaphragm 66′ is normally open, as shown in FIG. 6. The slit 68′ is closed by manual displacement of actuator 56′ from left to right into the position of FIG. 8.

Other than the reverse orientation of actuator 56′, the diaphragm 66′ and the normally open slit 68′, the components of FIGS. 6-9 is the same as the structure of the embodiment of FIGS. 1-5 and, therefore, no additional description of these components is necessary for one shifted in the art.

It is to be noted that when the diaphragm 66′ is stress free and at rest in the position of FIG. 6, flow through the normally open slit 68′ is accommodated. When the actuator 56′ is displaced from the position of FIG. 6 to the position of FIG. 8, the slit 68′ is closed. Thus, under the control of a medical technician, flow across diaphragm 66′ occurs in the position of FIG. 6 and is prevented in the position of FIG. 8. It is appropriate for the attendant to use the valve assembly of FIGS. 6-9 such that the diaphragm 66′ and the slit 68′ are utilized predominately in the position of FIG. 8 to prevent flow and only on occasions placed in the position of FIG. 6 to accommodate flow.

As can be seen best in FIGS. 1,3,5,7 and 9, actuators 56 and 56′ are of clamp shell design, comprising two halves held together by pins placed in holes 55 (FIG. 1) which are snapped or glued together. The two halves are contiguous at interfaces 53.

As can be seen from the Figures, slit 68 is depicted as being vertical and slit 68′ as horizontal. The slit orientation is simply selected to be compatible with the placement of the actuator 56, 56′.

The housing components 40, 42 and 54 and the actuator 56, 56′ may be of any suitable medical grade shape retaining synthetic resinous material heretofore used in valve control elements in the intravenous and respirator fields.

From FIGS. 3,5, 7 and 9 it is clear that surfaces 69 act as guide for cam 58 during operator displacement of actuators 56 and 56′ so that the actuators 56 and 56′ are lineally reciprocated.

If desired, the three housing members 40, 42 and 54 may be combined into a single one piece housing element, shown in FIG. 10 and generally designated 90. When a single piece housing 90 is utilized, it is presently preferred that the flanges at the proximal and distal ends of the elastomeric cannula 60 be somewhat enlarged as shown at 73′ in FIG. 10. Flange 73′ is placed in a somewhat undersized annular groove 92 in the housing by compressing the flange 73′ to cause it to be fitted into the annular groove 92, after which, the flange 73′ is allowed to expand to create a strong compression fit within the annular groove 92. If desired, a bonding compound may also be placed between the surface defining the groove 92 and the flange 73′.

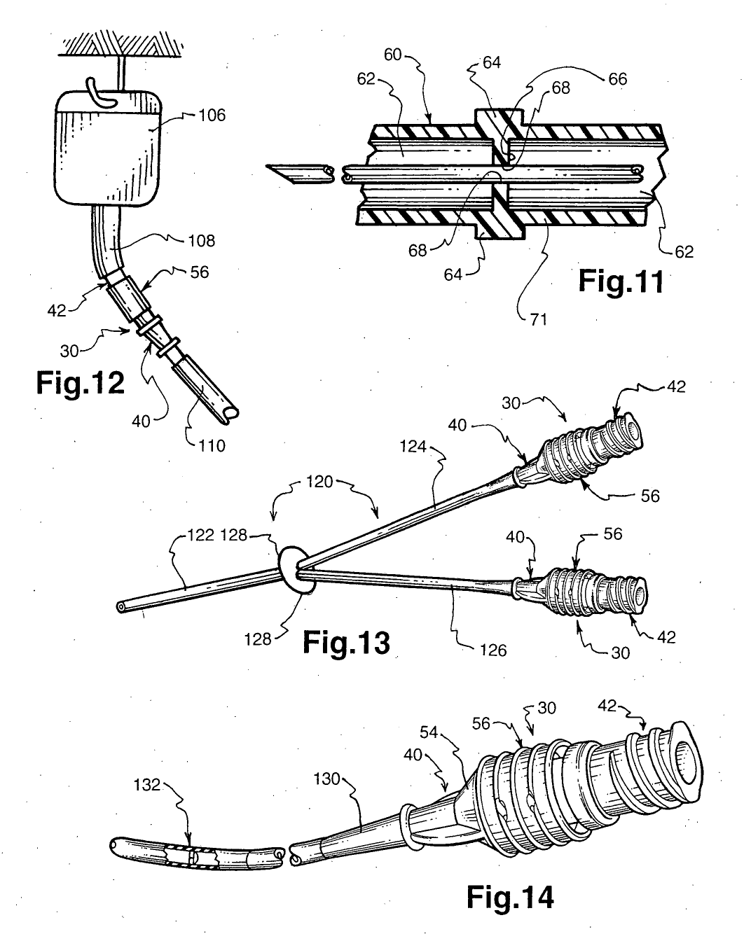

As shown in FIG. 11, the present invention also accommodates placement of hypodermic needle 104 directly through a normally open slit diaphragm 68, sized to insure that when the hypodermic needle 104 penetrates a body cavity, the normally open slit 68 does not accommodate fluid leakage along the needle and across the diaphragm 66. In addition, where the slit 68 is normally open, as shown in FIG. 11, it is not distorted by the needle but contiguously seals without distortion against the needle and, therefore, there is no leakage and no likelihood that slit 68 of FIG. 11 will take a set, making total closure of the slit difficult or impossible after removal of the needle.

While FIG. 11 illustrates use of a normally open slit, it is to be understood that the configuration of FIG. 12 could utilize a normally closed slit, where such is indicated to those skilled in the art.

FIG. 12 illustrates that the present invention may be utilized (in the form shown in FIG. 1) in conjunction with an IV bag 106 comprising a discharge tube 108 so as to be interposed between the discharge tube 108 and the traditional IV tubing 110 used to hydrate a medical patient with saline solution, among other things.

References now made to FIG. 13, which illustrates an additional form of the present invention. FIG. 13 illustrates a catheter assembly, generally designated 120 comprised of one distal catheter tube 122 and two proximal catheter tubes 124 and 126, which is representative of two or more proximal catheter tubes. Intravenous flow, for example, to the patient through catheter tube 122 may be solely fluid flow from catheter tube 124, or solely fluid flow from catheter 126, or merged flow from catheters 124 and 126. The side of the merger comprises taped down flaps or wings 128. Each of the two catheter tubes 124 and 126 has one slit valve assembly 30 connected to the proximal end of the catheter tube. Since the slit valve assembly 30 is described above, no further description is necessary for one skilled in the art based upon the version of the present invention as illustrated in FIG. 13.

FIG. 14 illustrates one way in which the present invention may be utilized at the proximal end of a catheter tube where the catheter tube is also equipped with a distal slit valve. Specifically, a catheter tube 130 is press fit, at its proximal end, over the distal end 50 of the housing member 40. The catheter tube 130 is also illustrated in FIG. 14 as comprising a distal slit valve 132, operated based on sufficient pressure differential, as is well-known in the catheter field. If proximal slit valve assembly 30 is normally closed and distal valve 132 normally closed, flow from the catheter tube 130 to the patient will occur only when the diaphragm of valve 30 is manually opened and pressure differential is high enough so that the slit of valve 132 is also open. If valve 30 comprises a normally open slit valve, left in the open position, then flow through the catheter tube 130 occurs when the pressure differential on the diaphragm of valve 132 is at or above the threshold value necessary for the slit of valve 132 to be open.

The invention may be embodied in other specific forms without departing from the spirit of essential characteristics thereof. The present embodiments therefore to be considered in all resects as illustrative and are not restrictive, the scope of the invention being indicated by the appended claims rather than by the foregoing description, and all changes which come within the meaning and range of equivalency of the claims are therefore intended to be embraced therein.

What is claimed is:

1. A medical valve assembly by which fluid flow is prevented and accommodated comprising:
a housing;
a valve actuator;
an elastomeric slit valving element defining a central lumen and comprising a slit diaphragm with memory and a diaphragm bridging across the lumen, the elastomeric valving element being concealed at least in part by the housing and the actuator such that responsive to manual manipulation the position of the valve actuator is changed causing the diaphragm of the elastomeric valving element to be either at rest or physically and forcibly displaced whereby the open and closed state of the slit is changed.

2. A medical valve assembly according to claim 1 wherein the housing is a multiple component assembly for outdwelling placement at the proximal end of a hollow medical cannula.

3. A medical valve assembly according to claim 1 wherein the valve actuator is displaceably carried by the housing and comprises an exposed gripping portion and a concealed radially inwardly extending portion axially manual movement of which accommodates diaphragm displacement.

4. A medical valve assembly according to claim 1 wherein the slit of the diaphragm is open when at rest and closed by the change in position of the valve actuator to physically and forcibly displace the elastomeric valving element.

5. A medical valve assembly according to claim 1 wherein the slit of the diaphragm is closed when at rest and open by the change in position of the valve actuator to physically and forcibly displace the elastomeric valving element.

6. A medical valve assembly according to claim 1 wherein the elastomeric slit valving element comprises an elastomeric sleeve with memory whereby the valve actuator when manually manipulated to change its position causes radially-directed force to be applied to the sleeve which is transferred to the diaphragm to open or close the slit.

7. A medical valve assembly according to claim 1 wherein the housing is selected from the group consisting of a one piece element and multiple piece elements.

8. A medical valve comprising:
a lumen-defining yieldable cannula of elastomeric material with memory;
a manually displaceable actuator located radially outward of the cannula;
a membrane comprising a slit bridging transversely across the lumen of the cannula, the slit being selected from the group comprising open slits when stress free and closed slits when stress free, the manual displacement of the actuator selectively radially displacing the cannula and the membrane to cause the slit to move between open and closed positions.

9. A slit valve comprising a lumen defining cannula of elastomeric material with memory comprising a membrane bridging across the lumen, having a normally open slit therein and the membrane actuator associated with the cannula by which radially force is supplied to the cannula to close the normally open slit.

10. A method of selectively accommodating medical patient flow of fluid through a slit valve, comprising the acts of:
manually displacing a valve actuator in respect to a lumen-defining elastomeric cannula with memory to respectively radially compress the cannula and place the cannula in a stress free condition;
causing the cannula when so compressed to displace a diaphragm bridging the lumen thereby shifting a slit in the diaphragm between open and closed positions.

11. A method according to claim 10 wherein the slit is normally open and the causing act closes the slit.

12. A method according to claim 10 wherein the slit is normally closed and the causing act opens the slit.

13. In combination:
tubing for connection to a medical patient by which a fluid is allowed to flow in respect to the medical patient;
a medical valve clamp associated with the tubing by which fluid flow with the tubing is prevented and accommodated comprising:
a housing;
a valve actuator;
an elastomeric slit valving element defining a central lumen and comprising a slit diaphragm with memory and a diaphragm bridging across the lumen, the elastomeric valving element being concealed at least in part by the housing and the actuator such that, responsive to manual manipulation, the position of the valve actuator is changed causing the diaphragm of the elastomeric valving element to be either at rest or physically and forcibly displaced whereby the open and closed state of the slit is changed.

14. A pre-packaged combination comprising:
a medical valve comprising;
a lumen-defining yieldable cannula of elastomeric material with memory;
a manually displaceable actuator located radially outward of the cannula;
a membrane comprising a slit bridging transversely across the lumen of the cannula, the slit being selected from the group comprising open slits when stress free and closed slits when stress free, the manual displacement of the actuator selectively radially displacing the cannula and the membrane to cause the slit to move between open and closed positions;
a needle assembled with the medical valve so as to extend through the slit;
packaging surrounding the assembled valve and needle.

15. A combination comprising:
a catheter tube having a proximal end and a distal region for use with a medical patient;
an outdwelling medical valve connected to the proximal end of the catheter tube comprising;
a lumen-defining yieldable cannula of elastomeric material with memory;
a manually displaceable actuator located radially outward of the cannula;
a membrane comprising a slit bridging transversely across the lumen of the cannula, the slit being selected from the group comprising open slits when stress free and closed slits when stress free, the manual displacement of the actuator selectively radially displacing the cannula and the membrane to cause the slit to move between open and closed positions;
an indwelling medical valve associated with the distal region.

16. A combination according to claim 15 wherein the indwelling medical valve comprises a slit valve.

17. A catheter assembly comprising at least two proximal catheter tubes which merge into one distal catheter tube;
a medical valve at the end of each proximal catheter tube comprising;
a lumen-defining yieldable cannula of elastomeric material with memory;
a manually displaceable actuator located radially outward of the cannula;
a membrane comprising a slit bridging transversely across the lumen of the cannula, the slit being selected from the group comprising open slits when stress free and closed slits when stress free, the manual displacement of the actuator selectively radially displacing the cannula and the membrane to cause the slit to move between open and closed positions.
